(19) United States
(12) Patent Application Publication
(10) Pub. No.: US 2011/0027189 A1
(43) Pub. Date: Feb. 3, 2011

(54) BIOCOMPATIBLE PRODUCTS FOR MAGNETIC PARTICLE IMAGING

(75) Inventors: Denis Markov, Veldhoven (NL); Hans Marc Bert Boeve, Hechtel-Eksel (BE)

Correspondence Address: PHILIPS INTELLECTUAL PROPERTY & STANDARDS, P.O. BOX 3001, BRIARCLIFF MANOR, NY, 10510, US

(73) Assignee: KONINKLIJKE PHILIPS ELECTRONICS N.V., EINDHOVEN (NL)

(21) Appl. No.: 12/935,018

(22) PCT Filed: Mar. 26, 2009
(86) PCT No.: PCT/IB09/51274

§ 371 Date: Sep. 28, 2010

(30) Foreign Application Priority Data
Apr. 3, 2008 (EP) 08103358.1

(51) Int. Cl. A61B 5/055; A61K 49/18; C12Q 1/02

(52) U.S. Cl. 424/9.3; 435/29

(57) ABSTRACT

The present invention relates to materials and methods for non-invasive imaging of biocompatible products. Biocompatible products are provided that can be visualized in vivo and in vitro using magnetic particle imaging methods. It was found that these materials can be employed for monitoring development, remodeling or degradation of biocompatible products. In another aspect, the inventive products enable substance delivery tracking and can be used to visualize targeted delivery of active agents.

### FIELD OF THE INVENTION

The present invention relates to materials and methods for non-invasive imaging of biocompatible products in vivo and in vitro.

### BACKGROUND OF THE INVENTION

Biocompatible products are employed in a multitude of biomedical applications. For example, biocompatible products include artificial tissue constructs, microcarriers or microcontainers as well as implants. However, to date, imaging techniques are rarely used for visualizing or monitoring, for example, remodelling, development or biodegradation of such products in vivo and in vitro. Computed tomography, magnetic resonance imaging (MRI) or ultrasound imaging have been suggested and can be utilized for such monitoring each having their disadvantages. Generally, contrast agents are used to increase the sensitivity of these techniques.

A method for monitoring remodelling of an artificial construct using signal enhancing agents and magnetic resonance imaging (MRI) is described in US 2006/0204445. However, contrast enhanced MRI senses a proton relaxation of water (rarely also lipids) in the magnetic field effected by a contrast agent. Therefore, only a contrast agent at the very interface between the biocompatible product and a body fluid or tissue will contribute to signal enhancement. This results in dramatic limitation of the signal to noise ratio. Alternatively, a contrast agent can be encapsulated with water in the bulk of the biocompatible product, which would improve the signal to noise ratio but would complicate a tissue construction and limit the number of its applications.

### SUMMARY OF THE INVENTION

In many biomedical applications it is essential to visualize biocompatible materials in vitro and in vivo.

One main field in biomedical applications regards tissue engineering which involves development of therapeutic strategies aiming at the replacement, repair, maintenance or enhancement of tissue function. For example, by seeding artificial tissue constructs such as scaffolds with cells, tissue is grown in vitro or in vivo to restore damaged tissue. During the process of tissue formation, the tissue is developed and then remodelled to become more like native tissue. However, a tissue-remodelling that takes place too slowly can lead to a pathologic response of surrounding tissues and compliance mismatch of the vessel, while rapid remodelling can result in premature failure of the engineered construct. Moreover, biodegradability is often an essential factor since artificial tissue constructs should preferably be absorbed by the surrounding tissue avoiding the necessity of a surgical removal. The rate at which degradation occurs has to coincide as much as possible with the rate of tissue formation. Thus, it would be advantageous to provide a material or method that allows to monitor development, remodelling and biodegradation of biocompatible materials such as artificial tissue constructs in vitro and in vivo.

A major problem in tissue engineering is the availability of a sufficient number of cells with the appropriate phenotype for delivery to damaged tissue. Commonly, this difficulty is overcome by using bioreactor culture systems for cell amplification which employ microcarriers and/or microcontainers. Besides serving as substrates for the propagation of anchorage-dependent cells, microcarriers and/or microcontainers can also be used to deliver the expanded undifferentiated or differentiated cells to the site of the defect or to deliver active agents in a living organism. However, the efficiency of the targeted delivery of cells or active agents depends greatly on the imaging procedures used. Therefore, it would be advantageous to provide a method for monitoring the targeted delivery of cells or active agents in vitro and in vivo. Furthermore, it would be advantageous to provide microcarriers and/or microcontainers that enable improved targeted delivery of cells or active agents to a target site.

Another class of widely used biocompatible products are implants. Especially in minimally-invasive surgeries, correct placement and orientation of the particular implant is difficult. Commonly, visibility of minimally-invasive procedures is accomplished through the use of endoscopes, or other such insertable magnification and/or illumination devices, wherein the image generated by an endoscope can be viewed through some type of ocular, or on a video display screen adjacent the surgery. Alternatively, many surgeries, especially graft implant procedures, utilize radiopaque markers on the implant and an external imager. Although great advances have been made in this respect, these systems are relatively expensive and all require sterilization or shielding in the operating room. Therefore, there is still a need for an improved method that enables monitoring of biocompatible materials after they have been introduced into a living organism. Furthermore, it would be advantageous to provide a biocompatible product that can be located or detected after it has been introduced in the desired target site.

To better address one or more of the above-mentioned needs or objects, in a first aspect of the invention a biocompatible product is provided that comprises at least one tracer agent suitable for magnetic particle imaging (MPI).

According to a further aspect of the invention, a method for visual monitoring a biocompatible product is provided, comprising the steps of providing a biocompatible product comprising the at least one MPI tracer agent, introducing the biocompatible product comprising the at least one MPI tracer agent to a target site, and detecting signals by magnetic particle imaging.

In another aspect of the invention an artificial tissue construct comprising at least one MPI tracer agent is provided.

In still another aspect of the invention a microcarrier comprising at least one MPI tracer agent is provided.

In still another aspect of the invention an implant comprising at least one MPI tracer agent is provided.

In still another aspect of the invention, the at least one MPI tracer agent is surrounded by a pharmaceutically acceptable shell.

The term “biocompatible product” as used herein refers to an artificial product that does not cause toxic or injurious effects on biological systems.

The term “scaffold” as used herein, means a material having an extended repeating structure, which forms a framework or matrix onto which and into which additional components may be introduced to impart additional features to the material.

The term “biodegradable” as used herein refers to a material that can be absorbed or degraded by a living organism.

As used herein, the term “microcarrier” refers to a rigid or semi-rigid, optionally porous particulate material used to support cell culture or as drug delivery system.

The term “microcontainer” as used herein refers to a rigid or semi-rigid, optionally porous material suitable to support cell culture, in which the cells can be shielded from the environment for at least some time during application such as cell handling or cell delivery.

The term “target site” is defined for the purposes of the present invention as the location, where the biocompatible product should have its effect, e.g. to replace damaged tissue. The target side can comprise a location in vitro, e.g., in a Petri dish, or in vivo, e.g. in a living organism.

The term “implant” as used herein refers to an object or material inserted or grafted into a living organism for prosthetic, therapeutic, diagnostic, or experimental purposes.

The term “MPI tracer agent” as used herein refers to a particulate material that can be employed in the magnetic particle imaging (MPI) method which is, for example, described in DE 10151778.

A “pharmaceutically acceptable shell” within the context of the present invention is a layer of a substance or a substance mixture which covers the core of the MPI tracer agent in such a way that, when administered to a patient, life-threatening side effects do not arise.

These and other aspects of the invention will be apparent from and elucidated with reference to the embodiments described hereinafter.

### DETAILED DESCRIPTION OF EMBODIMENTS

According to one embodiment of the present invention a biocompatible product is provided comprising at least one magnetic particle imaging (MPI) tracer agent. It has been found that enhanced or good signal-to-noise ratio can be obtained with magnetic particle imaging (MPI) techniques which are, for example, described in DE 10151778. In this method, a magnetic field having a spatial distribution of the magnetic field strength is generated such that a first sub-zone having a relatively low magnetic field strength and a second sub-zone having a relatively high magnetic field strength are formed in the examination area, wherein magnetic particles have been introduced into the examination area. The position in space of the sub-zones in the examination area is then shifted, so that the magnetization of the particles in the examination area changes locally. Signals are recorded which are dependent on the magnetization in the examination area, and by extracting information concerning the spatial distribution of the magnetic particles in the examination area an image thereof can be established. Thus, this method offers the possibility of non-invasive imaging with a high spatial resolution.

According to a further embodiment of the present invention, a method for visual monitoring a biocompatible product is provided, comprising the steps of providing a biocompatible product comprising the at least one MPI tracer agent, introducing the biocompatible product comprising the at least one MPI tracer agent to a target site, and detecting signals by magnetic particle imaging.

In one embodiment the biocompatible product is suitable for introduction into a living organism. The living organism can be a microorganism, a plant or a mammal, e.g., a human, monkey, dog, cat, mouse, rat, cow horse, pig, goat or sheep.

The biocompatible products of the present invention can be manufactured from any material that does not cause toxic or injurious effects on biological systems. Preferably, materials are used that are standard or commonly used materials for the respective biocompatible product. Examples include synthetic and natural polymers, precious metals, titanium and other metals, porcelain, alumina and other ceramics.

According to one embodiment, the biocompatible product is at least partially biodegradable.

Representative biodegradable organic materials may include natural or synthetic polymers, e.g., collagen, cellulose, silicone, poly(alpha esters) such as poly(lactate acid), poly(glycolic acid), polyorthoesters or polyanhydrides. In an exemplary embodiment the biodegradable material comprises a hydrogel. Hydrogels can be made biodegradable with a wide range of degradation times which may be useful, e.g., in tissue engineering, cell therapy applications or controlled release of pharmaceutically active agents.

Examples for biodegradable inorganic materials are metals or alloys based on at least one of magnesium or zinc, or alloys comprising at least one of Mg, Ca, Fe, Zn, Al, W, Ln, Si, or Y. Also suitable are, e.g., alkaline earth metal oxides or hydroxides, such as magnesium oxide, magnesium hydroxide, calcium oxide, and calcium hydroxide or mixtures thereof. In an exemplary embodiment of the invention, the biocompatible product can also comprise inorganic composites or organic composites or hybrid inorganic/organic composites.

In one embodiment, the biocompatible product is an artificial tissue construct. The artificial tissue construct can be, e.g., a scaffold providing a supportive framework that allows cells to attach to it, and grow to it. Typically, a scaffold can be placed in damaged tissue areas with the aim of inducing growth of cells from the surrounding healthy tissue to restore damaged tissue. In an exemplary embodiment the scaffold has a three-dimensional structure of interconnected pores to permit cell attachment.

Examples of synthetic polymers suitable for artificial tissue constructs comprise, e.g, poly(urethanes), poly(siloxanes) or silicones, poly(ethylene), poly(vinyl pyrrolidone), poly(2-hydroxy ethyl methacrylate), poly(N-vinyl pyrrolidone), poly(methyl methacrylate), poly(vinyl alcohol) (PVA), poly(acrylic acid), poly(vinyl acetate), polyacrylamide, poly(ethylene-co-vinyl acetate), poly(ethylene glycol), poly(methacrylic acid), polylactic acid (PLA), polyglycolic acids (PGA), poly(lactide-co-glycolides) (PLGA), nylons, polyamides, polyanhydrides, poly(ethylene-co-vinyl alcohol) (EVOH), polycaprolactone, poly(vinyl acetate), polyvinylhydroxide, poly(ethylene oxide) (PEO) and polyorthoesters or any combination thereof.

A commonly used synthetic biodegradable material for an artificial tissue construct is polylactic acid (PLA), a polyester which can degrade within the human body to form lactic acid, a naturally occurring chemical which can be easily removed from the body. Similar materials are polyglycolic acid (PGA) and polycaprolactone (PCL) which posses a degradation mechanism similar to that of PLA, but exhibit respectively a faster and a slower rate of degradation compared to PLA. The artificial tissue construct can also be made from natural materials, e.g. proteic materials, such as collagen or fibrin, and polysaccharidic materials, like chitosan or glycosaminoglycans (GAGs). Furthermore, the artificial tissue construct can comprise a decellularized matrix, i.e. a biostructure such as an organ or part thereof from which the cellular and tissue content has been removed by artificial methods leaving behind an intact acellular infrastructure. Decellularized matrices can be rigid, or semi-rigid, having an ability to alter their shape.

In another embodiment, the biocompatible product is a microcarrier. The microcarrier can be solid, e.g. to permit cell attachment only to the surface, or porous, e.g. to facilitate entrapment of cells in the interior. The microcarrier can have a spherical, non-spherical or irregular shape. Examples of suitable microcarriers may include microspheres, liposomes, and nanoparticles. In an exemplary embodiment, the microcarrier is spherical, and ranges in size from 1 to 300 μm. In another exemplary embodiment the microcarrier has a three-dimensional structure of interconnected pores, in order to increase the surface area available on which cells can grow. In still another exemplary embodiment the microcarrier comprises a functional multilayered structure. Alternatively or additionally, biologically active agents can be entrapped in the microcarrier and can be released in a living organism. The release of the active agent can happen immediately or in a controlled manner in vitro or in vivo.

The microcarrier can be manufactured from inorganic or organic materials. Suitable inorganic materials may include, e.g., calcium phosphates, calcium carbonates, calcium sulfates, glasses, hydroxylappatite, bioceramics or combinations of these materials. Organic materials may include, e.g., biopolymers such as collagen, gelatin, chitin, chitosan or chitosan derivatives, fibrin, dextran, agarose, or calcium alginate, particles of tissues such as bone or demineralized bone, cartilage, tendon, ligament, fascia, intestinal mucosa or other connective tissues, or chemically modified derivatives of these materials. Organic materials may also include synthetic polymeric materials, e.g., polylactic acid, polyglycolic acid, polycaprolactone, polycarbonates, polyesteramides, polyanhydrides, poly(amino acids), polyorthoesters, polyacetyls, polyacrylates, polycyanoacrylates, polyetheresters, poly(dioxanone)s, poly(alkylene alkylate)s, copolymers of polyethylene glycol and polyorthoester, polyurethanes, polystyrenes, polyvinyl chloride, polyvinyl fluoride, poly(vinyl imidazole), chlorosulphonate polyolefins, polyethylene oxide or polypeptides as well as blends and copolymers thereof. Examples for the manufacture of cross-linked dextran based microspheres are described by Hennink et al. (Biomacromolecules 2006, 7, 1983-2990, and Journal of controlled Release 2007, 122(1), 71-78).

In another embodiment the biocompatible product is a microcontainer. The microcontainer can permit cell attachment and can shield the cells from the environment for at least some time during application, e.g. cell handling or cell delivery. The microcontainer can be a planar, two-dimensional object (“flake”), wherein this object comprises a rigid or semi-rigid, optionally porous material which, upon application of an extrinsic stimulus, e.g. heating, change of pH or exposure to electromagnetic waves, is transferred from the planar state into a rolled state. The shape of the flake can resemble, e.g., a square, a rectangle or a parallelogram. Likewise, the flake can also have a circular, elliptic, trapezium like, hexagonal, polygonal or triangular shape. Typical dimensions of the flake are in the order of the dimensions of the cells or somewhat bigger than that, e.g., the size or the length of such a flake is from 10 μm to 100 mm. The thickness of the flake can be, e.g., from 100 nm to 1 mm. The rolled state can comprise, e.g., a bilayer, trilayer, multilayer or gradient structure. For example, the rolled state comprise an at least partially multilayered cylinder or a cylindrical body just substantially closed. Preferably, the cells are only present on the flakes and not on the substrate in between the flakes. However, it can also be desired that the cells are present in the interstitium between the flakes. The release of the cells inside a living organism can happen in a controlled way by controlling the transfer between the rolled and the planar state of the flake.

The microcontainer can be manufactured from a material which is biodegradable and biocompatible. For example, a hydrogel material may be suitable. Typical hydrogel materials may include, e.g., poly(meth)acrylic materials, substituted vinyl materials or mixtures thereof, as well as epoxydes, oxetans or thioles. Other suitable materials include, e.g., poly(glycolic acid), poly(lactic acid) and their copolymers and derivates. Other materials that can be employed can comprise, e.g., alginate, hyaluronic acid, chitosan, collagen, gelatin, silk or combinations thereof.

In a further embodiment the biocompatible product is an implant. Examples of implants may comprise, e.g., vascular endoprostheses, intraluminal endoprostheses, stents such as coronary stents or peripheral stents, surgical, dental or orthopedic implants, implantable orthopedic fixation aids, orthopedic bone prostheses or joint prostheses, artificial hearts or parts thereof, artificial heart valves, heart pacemaker casings or electrodes, subcutaneous and/or intramuscular implants, implantable drug-delivery devices, microchips, or implantable surgical needles, screws, nails, clips, or staples, or seed implants or the like. Typically, implants are made of solid materials, either polymers, ceramics or metals. To enable drug-delivery, implants can also be produced with porous surfaces or by using porous materials, wherein a drug may be included in the pore system for in vivo release.

Any suitable implant material may be used in the manufacture of the implants of the present invention. For example, the implant can be made from metal or metal alloys, e.g., selected from main group metals of the periodic system, transition metals such as copper, gold and silver, titanium, zirconium, hafnium, vanadium, niobium, tantalum, chromium, molybdenum, tungsten, manganese, rhenium, iron, cobalt, nickel, ruthenium, rhodium, palladium, osmium, iridium or platinum, or from rare earth metals. The material can also be made from organic materials, e.g., thermoset or thermoplastic polymers, synthetic rubbers, extrudable polymers, injection molding polymers, moldable polymers, spinnable, weaveable and knittable polymers, oligomers or pre-polymerizes forms or mixtures thereof. Suitable polymers include, e.g., hompopolymers, copolymers, prepolymeric forms and/or oligomers of poly(meth)acrylate, unsaturated polyester, saturated polyester, polyolefines like polyethylene, polypropylene, polybutylene, alkyd resins, epoxy-polymers or resins, phenoxy polymers or resins, phenol polymers or resins, polyamide, polyimide, polyetherimide, polyamideimide, polyesterimide, polyesteramideimide, polyurethane, polycarbonate, polystyrene, polyphenole, polyvinylester, polysilicone, polyacetale, cellulosic acetate, polyvinylchloride, polyvinylacetate, polyvinylalcohol, polysulfone, polyphenylsulfone, polyethersulfone, polyketone, polyetherketone, polybenzimidazole, polybenzoxazole, polybenzthiazole, polyfluorocarbons, polyphenylenether, polyarylate, cyanatoester-polymere, and mixtures thereof.

Magnetic particle imaging (MPI) allows direct 3D imaging of magnetic particles, i.e., MPI tracer agents. Within the context of the present invention, the term “magnetic particles” also refers to “magnetizable particles”. Spatial images are produced by measuring the magnetic fields generated by the MPI tracer agents introduced in an examination area. Magnetic particle imaging methods are disclosed, e.g., in DE 10151778 or US 2006/0210986. As described in said documents, it has been found that the change in the spatial distribution of the magnetic particles can be determined in the examination area, wherein the examination area can be present in living tissue or living organisms such as microorganisms, plants or mammals. For example, the change in the spatial distribution of the magnetic particles can be correlated with a local concentration, pressure, shear, viscosity, temperature and/or a local pH value. Furthermore, the particles are enriched to a different extent in different types of tissue. This effect enables so-called “molecular imaging” by visualizing the distribution of such particles and can be used, e.g., for monitoring development, remodeling or degradation of tissue.

MPI tracer agents which are suitable for the embodiments of the present invention are described, e.g., in WO 2004/091397, US 2007/0036729, DE 10151778 or DE 10238853. The MPI tracer agents may have a suitable size and shape. In an exemplary embodiment, the MPI tracer agent is dimensioned such that only a single magnetic domain (the monodomain) can form therein and there are no white regions. According to a one variant of the invention, suitable particle sizes lie in the range from 5 nm to around 5000 nm, 10 to 800 nm, or 20 to 50 nm. Typically, the upper limit depends on the material used.

For the embodiments of the present invention, it is a necessary prerequisite that the particles are biocompatible. Suitable materials of the MPI tracer agent can comprise magnetite (Fe₃O₄), maghemite (γ-Fe₂O₃) and/or non-stoichiometric iron oxides or mixtures thereof. In addition, further metal oxides can be added to the iron oxides, said metal oxides preferably being selected from magnesium, zinc and cobalt. These further metal oxides may be added to the iron oxide in proportions of up to 20% in total. Furthermore, manganese, nickel, copper, barium, strontium, chromium, lanthanum, gadolinium, europium, dysprosium, holmium, ytterbium and samarium can be contained in quantities of less than 5%, preferably less than 1%. However, any other suitable magnetic or magnetizable material can be used.

The biocompatibility of the MPI tracer agent can be further improved or modified by encapsulating the magnetic material. In one embodiment, the at least one MPI tracer agent can be surrounded by a pharmaceutically acceptable shell. In an exemplary embodiment, the substance or substance mixture the pharmaceutically acceptable shell is made of is biodegradable, and, e.g., can be cleaved into small units that can be used and/or can be removed by the living organism. A plurality of such substances and methods for producing them are described in the prior art, e.g., in U.S. Pat. No. 5,492,814 or US 2007/0036729. The pharmaceutically acceptable shell can comprise, e.g., synthetic polymers or copolymers, starch, dextran, cyclodextran, fatty acids, polysaccharides, lecithin, mono-, di- or triglycerides, proteins or polypeptides or a mixtures thereof.

In one embodiment, the at least one MPI tracer agent surrounded by a pharmaceutically acceptable shell further comprises at least one ligand which is immobilized on the surface of the shell. This can provide a considerable increase in affinity or specificity to the biocompatible material, in which the MPI tracer agent is incorporated or adhered to. By way of example, a polysaccharides, e.g. dextran, or polymer, e.g. polyvinyl alcohol, can be used as ligand.

In another embodiment of the present invention, the at least one MPI tracer agent can comprise a non-magnetic nucleus and a coating consisting of a magnetic material such as described above. In an exemplary embodiment, the non-magnetic nucleus can comprise physiologically acceptable natural materials, e.g., silica or latex.

The biocompatible material comprising at least one MPI tracer agent can be synthesized using any suitable method that is known in the art. Examples for applicable methods are nanofiber self-assembly, textile technologies such as wet or dry spinning methods, electro-spinning, knitting or weaving, as well as solvent casting, or emulsification/freeze-drying. Alternatively or additionally, suitable bulk materials may be structured in a suitable way by folding, embossing, punching, pressing, extruding, gathering, injection moulding and the like before or after being moulded or formed.

Textile technologies may include all the approaches that have been successfully employed for the preparation of non-woven meshes of different polymers. By way of example, it is refered to the process of electrospinning, which involves the creation of an electrical field at the surface of the liquid comprising at least one polymer. The resulting electrical force creates a jet of liquid which carries electrical charge. The liquid jets may be attracted to other electrically charged objects at a suitable electrical potential and as the jet of liquid elongates and travels, it will harden and dry. A number of examples for the manufacture of porous materials that are suitable, e.g. as artificial tissue constructs, using electrospinning can be found in US 2006/0204445.

In one embodiment, the at least one MPI tracer agent is incorporated in the biocompatible product. For example, the at least one MPI tracing agent may be embedded, entrapped or entangled within the biocompatible product or contained within cavities, enclosures, inclusions or pockets thereof. The at least one MPI tracer agent can be incorporated into the biocompatible product by mixing, blending or melding with the starting material employed in the corresponding method for manufacturing the biocompatible product. Alternatively, the biocompatible product can be loaded with the at least one MPI tracer agent subsequently, e.g., by dipping the biocompatible product into a bulk material containing the at least one MPI tracing agent.

In another embodiment, the at least one MPI tracer agent is adhered to the surface of the biocompatible product. For example, the at least one MPI tracer agent can be bound to a specific site of the biocompatible product. In an exemplary embodiment, the MPI tracer agent can be adsorbed to the surface of the biocompatible product. In another exemplary embodiment, covalent bonds can be formed between at least one ligand immobilized on a pharmaceutically acceptable shell of the at least one MPI tracing agent and functional groups on the surface of the biocompatible product.

In one embodiment, at least one MPI tracer agent is used for visual monitoring a biocompatible product by magnetic particle imaging. In an exemplary embodiment, the at least one MPI tracer agent is used for monitoring development, remodelling and degradation of an artificial tissue construct. For example, magnetic particle imaging (MPI) can be used for quantification of the local concentration of a tracer agent. Without wishing to be bound to any theory, the inventors believe that biodegradation and remodelling result in ‘ablation’, i.e. removal of the MPI tracer agent, which can be monitored by MPI signal reduction over time. Remodelling may also result in modification of the 3D image structure itself. The monitoring can be carried out in vitro or in vivo. For example, vascular and tissue organ constructs can be monitored.

In another exemplary embodiment, implants which have been functionalized or blended with at least one MPI tracer agent are visualized using magnetic particle imaging.

In another embodiment, the at least one MPI tracer agent is used for substance delivery tracking and monitoring. In an exemplary embodiment, the at least one MPI tracer agent is used for visualization of targeted delivery of active agents utilizing microcarriers loaded with active agents. In another exemplary embodiment, the at least one MPI tracer agent is used for monitoring tissue construction utilizing micro-carriers loaded with material for tissue engineering. Suitable therapeutic agents or materials for tissue engineering are, e.g., cells, biomedicals or pharmaceuticals. In still another embodiment, the at least one MPI tracer agent is used for visualization of targeted delivery of cells utilizing microcontainers loaded with cells.

While the present invention has been described in detail with respect to specific embodiments thereof, it should be noted that the above-mentioned embodiments are to be considered illustrative or exemplary and not restrictive; the invention is not limited to the disclosed embodiments. Variations to the disclosed embodiments can be understood and effected by those skilled in the art in practicing the claimed invention, from a study of the disclosure, and the appended claims. In the claims, the word “comprising” does not exclude other elements or steps, and the indefinite article “a” or “an” does not exclude a plurality. In the following, the invention is illustrated in view of an example. This example is, however, in no way meant to limit the invention as to its scope, but rather serve to illustrate the invention by way of one of its exemplary embodiments.

### EXAMPLES

### Example 1

### Manufacture of a Degradable Scaffold Containing a MPI Tracer Agent

45 wt.-% collagen I, 15 wt.-% elastin and 40 wt.-% poly(lactic-co-glycolic acid) (PLGA) are blended with iron oxide magnetic nanoparticles having a size of about 30 nm. The solutes are dissolved in 1,1,1,3,3,3-hexafluoro-2-propanol at a total concentration of 10 w/v % (100 mg/ml). The obtained solution is subjected to electospinning. A highly porous scaffold is obtained. High molecular weight PLGA can be added to the solution to increase mechanical strength of the scaffold. Scaffolds have typical thickness of 1 mm and lateral size of several centimeters.

### Example 2

### Manufacture of a Bilayer Microcontainer Containing a MPI Tracer Agent

Ebecryl 1810, supplemented with 0.1 wt % initiator (Irgacure 651) and 1:10 initiator inhibitor (4-methoxyphenol), is spincoated on a glass substrate (treated with 10 minutes UV-ozone) at 3000 RPM for 30 seconds. Subsequently the substrate is positioned beneath two masks, one squared and one striped, and irradiated for 1 second in the UV-setup UV-2 with filter. After rinsing with isopropylalcohol a pattern is obtained.

Subsequently, the glass plate with patterned Ebecryl layer is used as one of two substrates to make a cell as formed by two plan parallel substrates. The patterned Ebecryl layer is positioned on the inside of the cell. The spacing of the cell is set to be 50 μm by using 50 μm thick tape as spacers and the cell is filled via capillary forces with a reactive mixture consisting of 50 wt.-% n-isopropylacrylamide (NIPAA) (1 mol % diethyleneglycoldiacrylate (DEGDA)) blended with iron oxide magnetic nanoparticles having a size of about 30 nm in 50/50 wt.-% water/methanol. The cell is placed beneath a shadow mask (squared features) and irradiated with UV-light for 4 minutes. After irradiating the cell is opened by removing the top substrate and rinsed with water. Then the bottom substrate, containing the polymerized composite elements forming the flakes, is placed in a water bath at room temperature. At room temperature the PNIPAA hydrogel shows strong swelling in water and as a result the patterned flakes were released from the substrate and rolled up in the direction perpendicular to the Ebecryl 1810 stripes. Upon heating the water bath above 33° C. the PNIPAA hydrogel collapses (PNIPAA has a LCST at 33° C.) and the hydrogel layer shrinks. As a result, the bi-layers unrolled upon heating the water bath above 33° C.

1. A biocompatible product comprising at least one magnetic particle imaging (MPI) tracer agent.

2. The biocompatible product of claim 1, wherein the biocompatible product is an artificial tissue construct.

3. The biocompatible product of claim 1, wherein the biocompatible product is a microcarrier or a microcontainer.

4. The biocompatible product of claim 1, wherein the biocompatible product is an implant.

5. The biocompatible product of claim 1, wherein the at least one MPI tracer agent is incorporated into the biocompatible product.

6. The biocompatible product of claim 1, wherein the at least one MPI tracer agent is adhered to the surface of the biocompatible product.

7. The biocompatible product of claim 1, wherein the at least one MPI tracer agent is surrounded by a pharmaceutically acceptable shell.

8. The biocompatible product of claim 7, wherein the at least one MPI tracer agent surrounded by a pharmaceutically acceptable shell further comprises at least one ligand which is immobilized on the surface of the shell.

9. The biocompatible product of claim 1, wherein the at least one MPI tracer agent has a particle size between 5 and 5000 nm, preferably between 20 and 50 nm.

10. A method for visual monitoring a biocompatible product comprising at least one magnetic particle imaging (MPI) tracer agent, the method, comprising:
providing the biocompatible product comprising the at least one MPI tracer agent;
introducing the biocompatible product comprising the at least one MPI tracer agent to a target site; and
detecting signals arising from the at least one MPI tracer agent by magnetic particle imaging.

11. The method according to claim 10, wherein the step of detecting signals comprises the following steps:
localizing the signals;
quantifying the signals; and
evaluating the magnetic particle imaging data obtained for the biocompatible product.

12. A method comprising use of an MPI tracer agent for visual monitoring a biocompatible product comprising at least one magnetic particle imaging (MPI) tracer agent using magnetic particle imaging.
